(19) United States
(12) Patent Application Publication
(10) Pub. No.: US 2020/0194034 A1
(43) Pub. Date: Jun. 18, 2020

(54) Medical Image Management Apparatus and Medical Image Management System

(71) Applicant: Konica Minolta, Inc., Chiyoda-ku (JP)

(72) Inventors: Eiichi AKAGI, Hachioji-shi (JP); Yasuhiro Saiki, Shibuya-ku (JP)

(21) Appl. No.: 16/714,334

(22) Filed: Dec. 13, 2019

(30) Foreign Application Priority Data
Dec. 18, 2018 (JP) 2018-235976

(51) Int. Cl. G11B 27/19; G06T 7/246; G06T 7/00; G06T 11/20; G16H 30/40; A61B 6/00; G11B 27/031

(52) U.S. Cl. CPC G11B 27/19; G06T 7/246; G06T 7/0016; G06T 11/206; G06T 2207/30061; A61B 6/5205; A61B 6/5294; A61B 6/50; G11B 27/031; G16H 30/40

(57) ABSTRACT

A medical image management apparatus comprising a first hardware processor that: acquires a first video obtained by radiographing a cyclic motion of a subject; acquires a second video obtained by radiographing a cyclic motion of a subject separately from the radiographing of the first video; extracts a first feature that is a feature of a cyclic change of the acquired first video; extracts a second feature that is a feature of a cyclic change of the acquired second video; adjusts at least one of the first video and the second video so that a cycle of change approaches the cycle of change of the other video, based on the extracted first feature and second feature; and outputs the acquired first video or the adjusted first video, and the acquired second video or the adjusted second video.

### CROSS-REFERENCE TO RELATED APPLICATIONS

The entire disclosure of Japanese Patent Application No. 2018-235976 filed on Dec. 18, 2018 is incorporated herein by reference in its entirety.

### BACKGROUND

### Technological Field

The present invention relates to a medical image management apparatus and a medical image management system.

### Description of the Related Art

Conventionally, video obtained by radiographing a subject's cyclic movements (for example, breathing) has been accumulated in a picture archiving and communication system (hereinafter referred to as PACS) so that the accumulated video is used later to diagnose the subject. This is the case when a diagnosis is made while the video of the subject is viewed alone or when a diagnosis is made while the video is compared with a previous video of the subject or a video of a subject different from that subject.

However, respiratory cycles are often different between a subject to be diagnosed and a subject who is different from that subject, or between the current subject to be diagnosed and the previous subject; therefore, when videos are played back side by side, the subject's conditions in the videos at the same time may deviate from each other due to the difference between the motion cycles of the videos, which may make it difficult to make a diagnosis while comparing them.

In view of this, as described in Japanese Patent Laid-Open No. 2018-138193, a technique has been proposed in which the time of presentation is changed depending on each frame image constituting at least one of the reference video and the referential video to match the speeds of a first cyclic change and a second cyclic change.

### SUMMARY

However, in order to use the function for matching the motion cycles of multiple videos described above, the image processing device described in Japanese Patent Laid-Open No. 2018-138193 should conventionally be added to a system for managing medical images (the dynamic image radiographing system in Japanese Patent Laid-Open No. 2018-138193).

This complicates the system configuration and makes the system expensive.

An object of the present invention is to achieve adjustment of the change cycles in multiple videos obtained by radiographing cyclic motions of a subject, with a simpler configuration.

To achieve at least one of the abovementioned objects, according to an aspect of the present invention, a medical image management apparatus reflecting one aspect of the present invention comprises: a first hardware processor that acquires a first video obtained by radiographing a cyclic motion of a subject; acquires a second video obtained by radiographing a cyclic motion of a subject separately from the radiographing of the first video; extracts a first feature that is a feature of a cyclic change of the acquired first video; extracts a second feature that is a feature of a cyclic change of the acquired second video; adjusts at least one of the first video and the second video so that a cycle of change approaches the cycle of change of the other video, based on the extracted first feature and second feature; and outputs the acquired first video or the adjusted first video, and the acquired second video or the adjusted second video.

To achieve at least one of the abovementioned objects, according to another aspect of the present invention, a medical image management system reflecting one aspect of the present invention comprises: a medical image management apparatus including a first hardware processor that acquires a first video obtained by radiographing a cyclic motion of a subject, acquires a second video obtained by radiographing a cyclic motion of a subject separately from the radiographing of the first video, extracts a first feature that is a feature of a cyclic change of the acquired first video, extracts a second feature that is a feature of a cyclic change of the acquired second video, adjusts at least one of the first video and the second video so that a cycle of change approaches the cycle of change of the other video, based on the extracted first feature and second feature, and outputs the acquired first video or the adjusted first video, and the acquired second video or the adjusted second video; and a display apparatus including a display and a second hardware processor that causes the display to show the first video acquired by the medical image management apparatus or the first video adjusted by the medical image management apparatus, and the second video acquired by the medical image management apparatus or the second video adjusted by the medical image management apparatus.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 is a block diagram showing a medical image management system according to an embodiment of the present invention;

FIG. 2 is a block diagram showing a medical image management apparatus provided in the medical image management system shown in FIG. 1;

FIG. 3 is a flowchart showing a diagnostic preparation process executed by the medical image management apparatus shown in FIG. 2;

FIG. 4 is a block diagram showing a medical image display apparatus provided in the medical image management system shown in FIG. 1;

FIG. 5 is a diagram showing an example of an image confirmation screen displayed on the medical image display apparatus shown in FIG. 4;

FIG. 6 is a diagram showing another example of an image confirmation screen displayed on the medical image display apparatus shown in FIG. 4;

FIG. 7 is a diagram for explaining a method of displaying a first partial video and a second partial video on the medical image display apparatus shown in FIG. 4;

FIG. 8 is a diagram for explaining a method of displaying a first partial video IR1 and a second partial video IR2 on the medical image display apparatus shown in FIG. 4;

FIG. 9 is a diagram for explaining a method of readjusting a video using the medical image display apparatus shown in FIG. 4;

FIG. 10 is a diagram for explaining another method of readjusting a video using the medical image display apparatus shown in FIG. 4;

FIG. 11 is a diagram showing an example of a second graph displayed on the medical image display apparatus shown in FIG. 4; and

FIG. 12 is a diagram showing an example of a second graph displayed on the medical image display apparatus shown in FIG. 4.

### DETAILED DESCRIPTION OF THE EMBODIMENTS

An embodiment of the present invention will now be described with reference to the accompanying drawings. However, the scope of the invention is not limited to the disclosed embodiments.

### [Radiographic Imaging System]

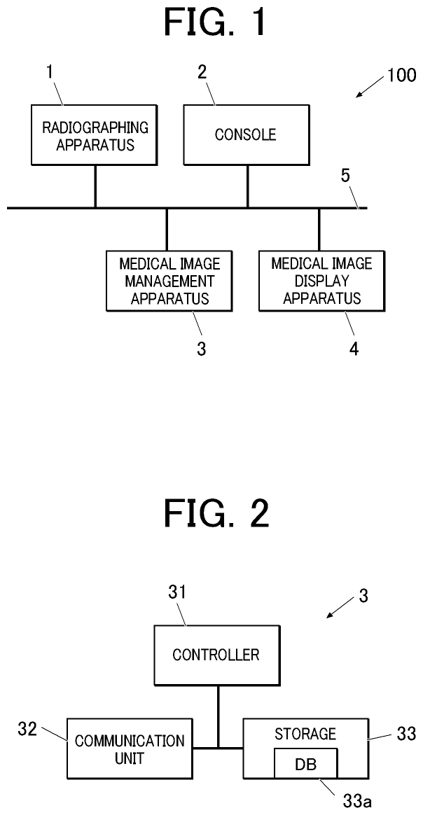

First, a schematic configuration of a medical image management system 100 according to this embodiment will be described. FIG. 1 is a block diagram showing the medical image management system 100.

As shown in FIG. 1, the medical image management system 100 according to this embodiment includes a radiographing apparatus 1, a console 2, a medical image management apparatus 3, and a medical image display apparatus 4.

These apparatuses 1 to 4 can communicate with each other via a communication network 5.

In addition, the medical image management system 100 is connectable to a hospital information system (HIS), a radiology information system (RIS), a medical image analysis apparatus, and the like not shown in the drawings.

The radiographing apparatus 1 may be any apparatus that can generate a video having multiple frames that can be adjusted by the medical image management apparatus 3 which will be described later, and can be, for example, a radiographing apparatus that generates radiographs according to radiation generated by a radiation generating apparatus, or an optical camera.

Note that the radiographing apparatus 1 may be capable of radiographing still images in addition to videos.

Radiographing apparatus 1 may be connected to the communication network 5 not directly but via the console 2, the medical image management apparatus 3, or the like.

A video may be transferred from the radiographing apparatus 1 to another apparatus not via the communication network 5 but using a storage medium or a cable.

The console 2 is composed of a PC or a dedicated apparatus.

Through the console 2, various radiographing conditions (for example, tube voltage, tube current, irradiation time (mAs value), and frame rate) can be set for the radiographing apparatus 1 or the like according to inspection information acquired from other apparatuses (for example, HIS and RIS) and operations by the operator.

The medical image management apparatus 3 constitutes a picture archiving and communication system (PACS), and includes a PC, a dedicated apparatus, or a virtual server on the cloud.

The details of the medical image management apparatus 3 will be described later.

The medical image display apparatus 4 in this embodiment is composed of a PC or a portable terminal, a dedicated device, and the like.

The details of the medical image display apparatus 4 will be described later.

Note that the medical image display apparatus 4 is not necessarily provided and the console 2 may be used in place of the medical image display apparatus 4 or a display may be provided to the medical image management apparatus 3.

### [Medical Image Management Apparatus]

Next, a specific configuration of the medical image management apparatus 3 provided in the medical image management system 100 will be described. FIG. 2 is a block diagram showing the medical image management apparatus 3.

As shown in FIG. 2, the medical image management apparatus 3 according to this embodiment includes a controller 31 (first hardware processor), a communication unit 32, and a storage 33.

The controller 31 includes a central processing unit (CPU) and a random access memory (RAM). The CPU of the controller 31 reads various programs stored in the storage 33 and expands them in the RAM, executes various processes according to the expanded programs, and centrally controls the operations of the components of the medical image management apparatus 3.

The communication unit 32 consists of a wireless module and the like and can transmit and receive various signals and various medical images (for example, still images, videos, and analysis videos) to/from other apparatuses and the like 1, 2, and 4 via the communication network 5 (for example, a local area network (LAN), a wide area network (WAN), or the Internet).

The storage 33 consists of a non-volatile semiconductor memory or a hard disk, and stores various programs to be executed by the controller 31, parameters necessary for execution of the programs, and the like.

In addition, the storage 33 includes a database 33a for accumulating various medical images.

Note that the database 33a may be provided not in the storage 33 but in an apparatus different from the medical image management apparatus 3.

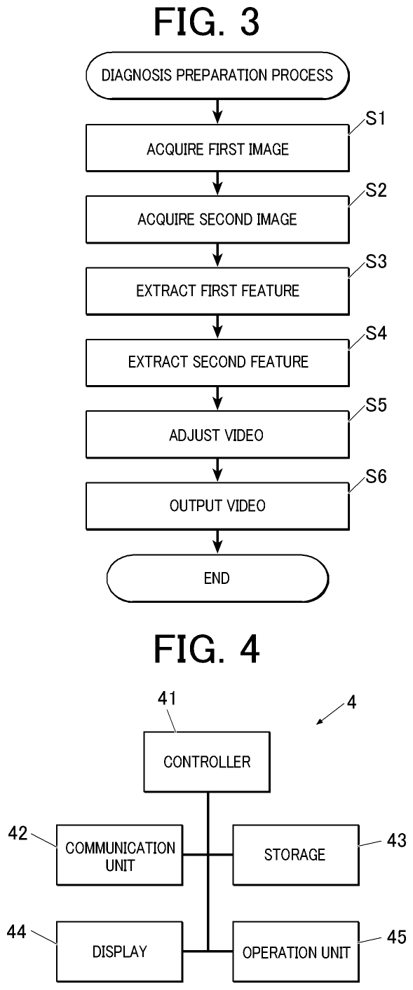

The controller 31 of the medical image management apparatus 3 with such a configuration has a function of executing the diagnostic preparation process shown in FIG. 3, for example, upon transmission of a video from the radiographing apparatus 1 or the console 2 or when a predetermined operation has been performed on any of the apparatuses 1 to 4.

To be specific, the controller 31 first acquires data of the first video (Step S1). This “first video” is the main video used for diagnosis and is obtained by radiographing the subject's cyclic motions.

In addition, the “first video” is mainly, for example, a video in which multiple frames are combined, the frames being obtained by repeatedly radiographing a human chest or the like in a short time with the radiographing apparatus 1 which is used in combination with a radiation generating apparatus.

“A cyclic motion” includes lung expansion/contraction, diaphragm ascent/descent, heart expansion/contraction, and joint bending/extension.

Note that the first video may be an original video generated by the radiographing apparatus 1 or an analyzed video obtained by analyzing the original video with a medical image analysis apparatus (not shown).

This “analysis” includes a process for emphasizing a structure, a process for attenuating a structure (for example, bone), a process for visualizing a movement of expiratory ventilation made from a difference image between multiple frames when the target is a living body, and a process for visualizing a movement of blood flow, and is also referred to as dynamic analysis.

In this embodiment, video data according to an image request signal transmitted from the medical image display apparatus 4 which will be described later is acquired as data of the first video. When the medical image management apparatus 3 includes an operation unit, a video according to an operation performed on the operation unit may be acquired as the data of the first video.

The data of the first video is acquired when it is received from another apparatus (for example, the radiographing apparatus 1 or the console 2) via the communication unit 32 or when it is called from the database 33a.

When the data of the first video is received from another apparatus, the controller 31 accumulates it in the database 33a. The accumulated data of the first video can be used as the data of the second video when another data set of the first video is acquired.

After acquisition of the data of the first video, the controller 31 acquires the data of the second video (Step S2).

This “second video” is a comparative video to be compared with the first video, and is obtained by radiographing a cyclic motion of a subject in addition to radiographing the first video.

The second video in this embodiment is obtained by radiographing the same subject as the subject of the first video in the past, or is obtained by radiographing a subject who is different from the subject of the first video and has a similar physique, a similar age, or the same gender to/as that subject.

This “similar physique” refers to, for example, a physique having the same or a close vertical or horizontal width of the lung field region obtained by analyzing video data.

Like the first video, the second video may be an original video generated by the radiographing apparatus 1 or an analyzed video obtained by analyzing the original video with a medical image analysis apparatus (not shown).

Multiple pieces of data of the second video may be acquired for one piece of data of the first video.

In this embodiment, video data according to an image request signal transmitted from the medical image display apparatus 4 which will be described later or the data of a video automatically selected based on the data of the acquired first video is acquired as the data of the second video.

At that time, whether or not the data of the video to be acquired matches the data of the second video, that is, whether or not the subject of the second video is the same as the subject of the first video, whether or not the subject of the second video is at a similar age to the subject of the first video, whether or not the subject of the second video has the same gender as the subject of the first video, or whether or not the subject of the second video has a similar physique to the subject of the first video is determined, for example, based on inspection information or subject information in the data of the first video and the data of the second video, or the width of the rib cage that appears in the video.

Such a measure allows the user to acquire the second video related to the first video without taking time and effort.

If the first video is selected at this time, candidates of the second video may be displayed to be selected by the user.

As in the case of the data of the first video, the data of the second video is acquired when it is received from another device via the communication unit 32 or when it is called from the database 33a.

When the data of the second video is received from another apparatus 1 or 2, the controller 31 accumulates it in the database 33a.

In this embodiment, the process of Step S2 is carried out after Step S1; alternatively, the process of Step S2 may be carried out before Step S1 or concurrently with Step S1.

After acquisition of the data of the first video, the controller 31 extracts a first feature (Step S3).

The “first feature” is, for example, a feature of a cyclic change of the acquired first video, and specifically indicates the cycle or phase of change or the like.

The method described in Patent Literature 1, for example, can be used for the extraction of the first feature.

After acquisition of the data of the second video, the controller 31 extracts a second feature (Step S4).

Like the first feature, the “second feature” is, for example, a feature of a cyclic change of the acquired second video, and specifically indicates the cycle or phase of change or the like.

A method described in Patent Literature 1, for example, can be used for the extraction of the second feature.

The process of Step S4 is carried out after Step S3 here; alternatively, the process of Step S4 may be carried out before Step S3 or concurrently with Step S3.

After the first and second features are extracted, the controller 31 adjusts the data of the second video so that the cycle of change of the second video approaches the cycle of change of the first video, according to the extracted first feature and second feature (Step S5).

Here, the reason for making adjustment so that the cycle of change of the second video is brought close to the cycle of change of the first video is that the target of diagnosis is the first video and bringing the first video in a state close to the radiographed state enables a more accurate diagnosis.

A method described in Patent Literature 1, for example, can be used for the adjustment of the data of the second video.

It should be noted that the phases of the first video and the second video are not necessarily completely matched, and may be shifted within a range of, for example, a few frames (such a range that the diagnostician cannot recognize the shift between the two videos by the naked eye).

Even for the first video, a slight adjustment cannot be a big problem or sometimes diagnosis is intentionally made based on the state of the comparative second video; therefore, adjustment of the first video is not necessarily prohibited and no problems arise if a function of adjusting the first video may is given.

In this embodiment, the portion of the second video in which a change in the second or later period is reflected is adjusted.

A change in the first period is not always a cyclic change depending on the timing of the start of radiographing and is sometimes difficult to adjust, but the change in the second or later period tends to be a cyclic change; thus, adjustment can be easily performed.

In this embodiment, the cycle of the second video, which is a comparative video, is brought close to the cycle of the first video, which is the main video. Alternatively, the data of the first video may be adjusted, or both the data of the first video and the data of the second video may be adjusted.

If there is no difference between the first and second features extracted in Steps S3 and S4, or the difference is within a negligible range, the process of Step S5 may be skipped and may jump to Step S6.

After adjustment of the video data, the controller 31 outputs the acquired data of the first video and the acquired data of the second video or the adjusted data of the second video (Step S6).

In this embodiment, the data of the first video and the data of the second video are transmitted to the medical image display apparatus 4 via the communication unit 32. Alternatively, they may be output via a storage medium or a cable.

In the case where the medical image management apparatus 3 is provided with a display, to output may refer to presentation on the display.

The controller 31 has the following functions in addition to the function of carrying out the diagnosis preparation process.

For example, the controller 31 has the function of generating a statistical value based on multiple second videos accumulated in the database 33a.

This statistical value is for displaying second and third graphs G2 and G3, which will be described later, in the medical image display apparatus 4. In this embodiment, the average of each cycle of multiple second videos is calculated based on the second features extracted from the respective pieces of data of the multiple second videos, or an upper limit signal value corresponding to a predetermined upper limit reference and a lower limit signal value corresponding to a predetermined lower limit reference are extracted from a distribution of predetermined signal values extracted from the multiple second videos. In the case where a probability distribution is used, the upper probability is the upper limit reference and the lower probability is the lower limit reference.

In addition, the controller 31 in this embodiment can readjust the data of the second video.

To be specific, the cycle of change of the second video is readjusted in accordance with a readjustment command signal (the amount of transformation of the waveform of the second graph), which will be described later, transmitted from the medical image display apparatus 4.

This readjustment command signal is transmitted when the user performs a predetermined operation on the medical image display apparatus 4. A specific operation performed by the user for readjustment will be described later.

This settles the problem when the initial adjustment is insufficient or when the video fluctuates irregularly.

In this embodiment, the data of the second video that is the comparative video is adjustable and the data of the second video is therefore readjustable. Alternatively, in the case where the data of the first video or both the data of the first video and the data of the second video is adjustable, the data of the first video may be readjustable, or both the data of the first video and the data of the second video may be readjustable.

### [Medical Image Display Apparatus]

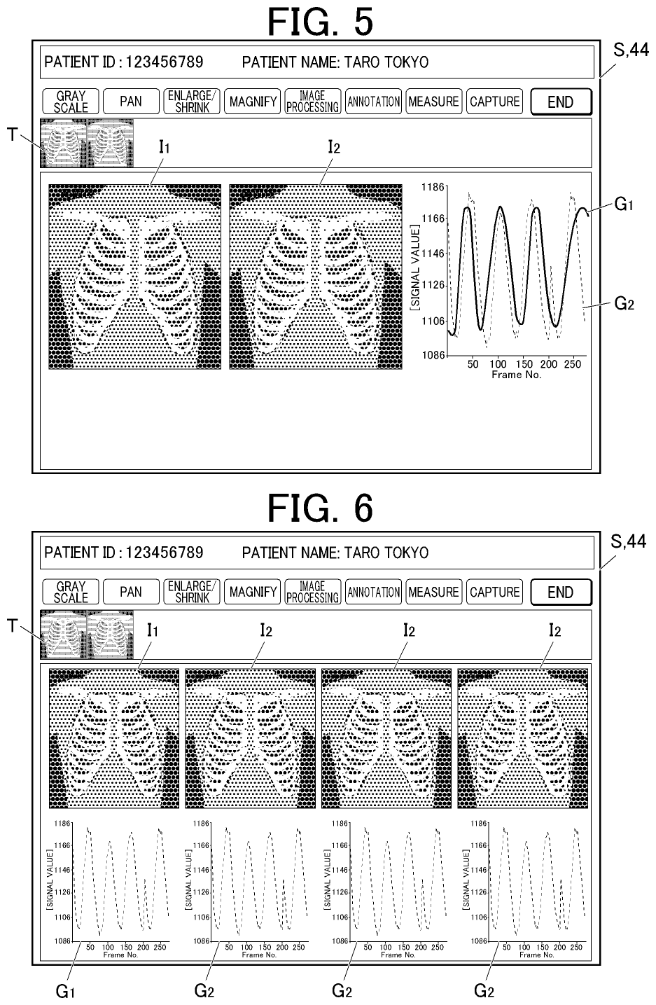

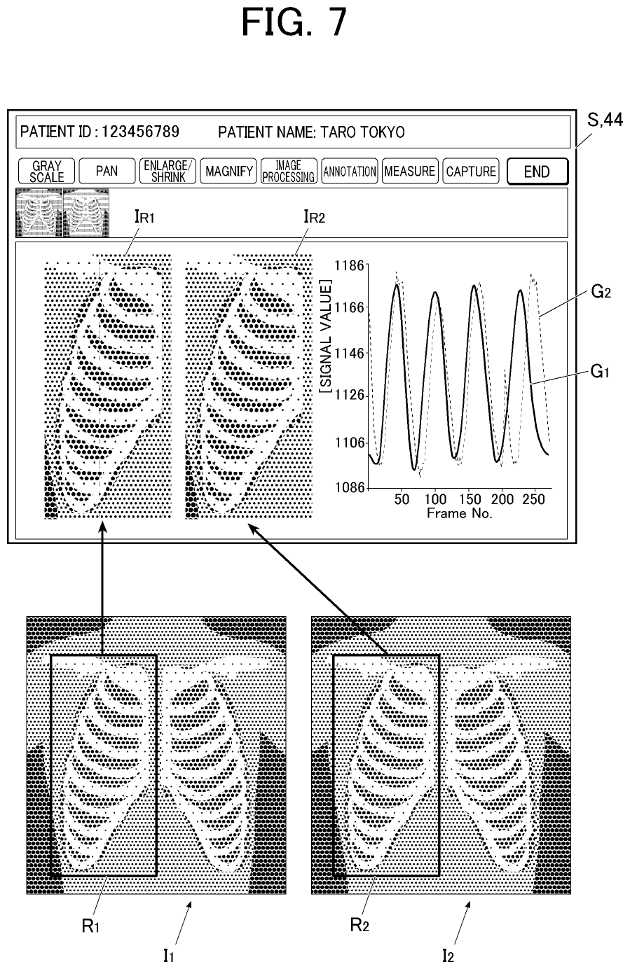

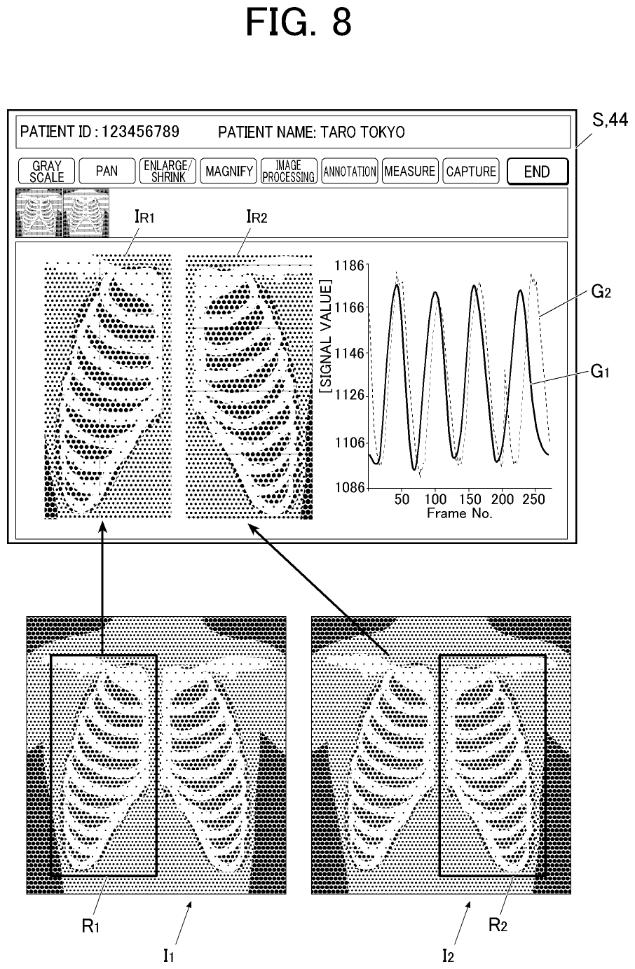

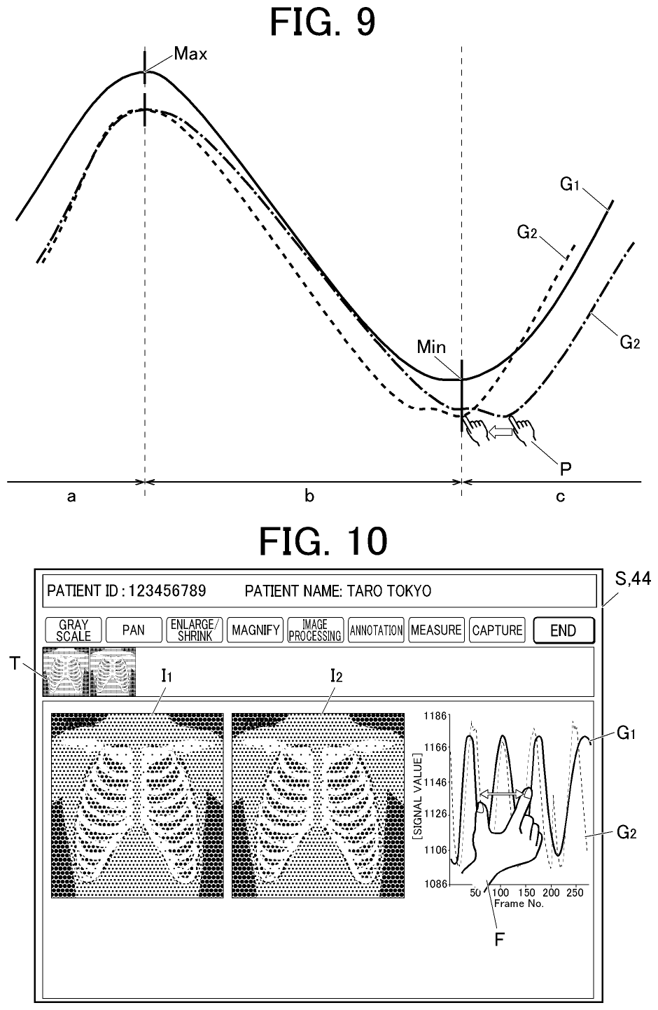

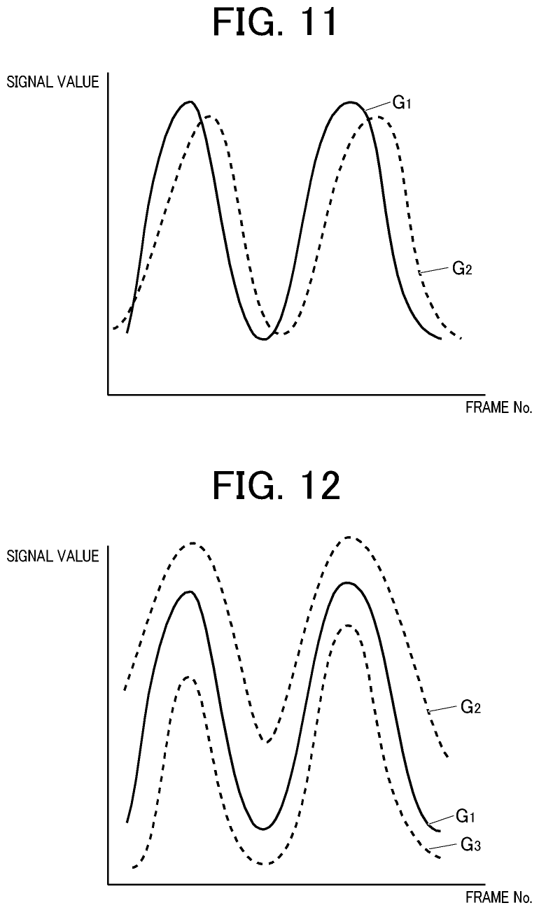

Next, a specific configuration of the medical image display apparatus 4 provided in the aforementioned medical image display system will be described. FIG. 4 is a block diagram showing the medical image display apparatus 4, FIGS. 5 and 6 are diagrams showing examples of image confirmation screen S displayed on the medical image display apparatus 4, FIGS. 7 and 8 are diagrams for explaining a method of displaying the first partial video IR1 and the second partial video IR2 on the medical image display apparatus 4, FIGS. 9 and 10 are diagrams for explaining a method of readjusting the data of a video using the medical image display apparatus 4, and FIGS. 11 and 12 are diagrams showing an example of the second graph G2 displayed on the medical image display apparatus 4.

As illustrated in FIG. 4, the medical image display apparatus 4 according to this embodiment includes a controller 41 (second hardware processor), a communication unit 42, a storage 43, a display 44, and an operation unit 45.

The controller 41 and the communication unit 42 have the same configurations as the controller 31 and the communication unit 32 of the medical image management apparatus 3.

The storage 43 consists of a nonvolatile semiconductor memory, a hard disk, or the like, and stores various programs to be executed by the controller 41, parameters necessary for executing the programs, and the like.

The display 44 consists of a monitor such as a liquid crystal display (LCD) or a cathode ray tube (CRT), and shows various images, information, and the like according to instructions given by display signals input from the controller 41.

The operation unit 45 consists of a keyboard having cursor keys, numeric input keys, various function keys, and the like, a pointing device such as a mouse, a touch panel stacked on a surface of the display 44, or the like so as to be able to be operated by the operator.

Note that the medical image display apparatus 4 is not necessarily provided with the operation unit 45, and the console 2, the medical image management apparatus 3, or the like may be provided with an operation unit.

The controller 41 of the medical image display apparatus 4 configured as described above has the following functions.

For example, the controller 41 has a function of requesting video data to be displayed on the display 44.

In this embodiment, an image request signal dependent on the operation on the operation unit 45 (for example, an inspection or thumbnail selected from an inspection list or multiple video thumbnails T displayed on the display 44) is transmitted to the medical image management apparatus 3 via the communication unit 42.

The controller 41 has the function of acquiring the data of a first video I1 transmitted from the medical image management apparatus 3 and the data of a second video I2 or the adjusted data of the second video I2.

In this embodiment, the data of the first video I1 and the data of the second video I2 are received via the communication unit 42. Alternatively, they may be acquired via a storage medium or a cable.

Further, as shown in FIG. 5, for example, the controller 41 has the function of causing the display 44 to display the first video I1 and the second video I2 or the adjusted second video I2.

Since the data of the first video I1 and the data of the second video I2 acquired by the medical image display apparatus 4 have the same cycle from the beginning or have been adjusted by the medical image management apparatus 3, the display 44 displays the first video I1 and the second video I2 that have the same cycle and are in phase.

When the display mode (for example, stop, fast forward, fast reverse, or slow playback) of the first video I1 is changed, the display mode of the second video I2 may be changed in accordance with the first video I1.

FIG. 5 shows the case where both the first video I1 and the second video I2 are displayed; however, either the first video I1 or the second video I2 may be displayable depending on the case.

Further, as shown in FIG. 5, the controller 41 has the function of causing the display 44 to display the first graph G1 and the display 44 to display the second graph G2.

The “first graph G1” is a graph representing the relationship between the time (frame number) and the signal value in the first video I1. In this embodiment, the horizontal axis represents the frame number, and the vertical axis represents the signal value.

The “second graph G2” is a graph representing the relationship between the time (frame number) and the signal value based on the second video I2. In this embodiment, as in the first graph G1, the horizontal axis represents the frame number, and the vertical axis represents the signal value.

FIG. 5 shows the case where both the first graph G1 and the second graph G2 are displayed; however, either the first graph G1 or the second graph G2 may be displayable depending on the case.

FIG. 5 shows the case where the first and second graphs G1 and G2 are displayed with the first and second videos I1 and I2; however, only the first graph G1 or the second graph G2 may be displayable depending on the case.

FIG. 5 shows the case where the first and second graphs G1 and G2 are displayed, being superimposed on each other; however, as shown in FIG. 6, they may be displayed for each of the videos I1 and I2.

In addition, as shown in FIG. 7, for example, the controller 41 in this embodiment can extract a partial region in each frame of the first video I1 as a first region R1 and cause the display 44 to show a first partial video IR1 that shows only the first region R1, and extract a partial region, which corresponds to the first region R1, in each frame of the second video I2 as a second region R2 and cause the display 44 to show a second partial video IR2 that shows only the second region R2.

For example, in the case where the first video I1 and the second video I2 are radiographic videos obtained by radiographing the chest, settings can be made so that one of the left and right lungs is the first region R1, and the lung on the same side as the first region R1 shown in FIG. 7 or the lung on the opposite side to the first region R1 shown in FIG. 8 is the second region R2.

In addition, the controller 41 has the function of transforming the waveform of the second graph G2 in accordance with an operation performed on the operation unit 45, that is, the function of receiving a user's operation for performing readjustment of the graph.

The second graph G2 displayed on the display 44 may become, for example, like that indicated by the dashed-dotted line in FIG. 9 due to insufficient adjustment of the second video I2 in the medical image management apparatus 3. In other words, the frame with the maximum Max (peak) almost coincides with the first graph G1, but the frame with the minimum Min (valley) may shift to the right with respect to the first graph G1. This shows that the cycle of the second video I2 after adjustment is still longer than the cycle of the first video I1.

Therefore, in this embodiment, for example, the display region of the graph is divided into the region from the maximum Max to the minimum Min, and the region from the minimum Min to the maximum Max. When the frames with the maximum match and the frames with the minimum do not match as shown in FIG. 9, the maximum Max is fixed by clicking with a pointer P positioned in the region c that is on the right with respect to the minimum Min. Dragging the pointer P to the left or right in this state enlarges or shrinks the second graph G2 only in the width direction, leaving the maximum Max in the same position. In other words, in the case shown in FIG. 9, dragging the pointer P to the left forms the second graph G2 into the waveform indicated by the dashed line, thereby matching the minimum Min with Min of the first graph G1.

Although not shown in the drawings, clicking the pointer P in the region a which is on the left with respect to the maximum Max fixes the minimum Min and enlarges or shrinks the second graph G2 only in the width direction, leaving the minimum Min in the same position. Clicking in the region b which is between the maximum Max and the minimum Min causes the waveform of the second graph G2 to move in parallel without changing.

Aside from these operations, clicking somewhere in the second graph G2 may display a rectangular frame enclosing the second graph G2, and dragging the vertical line to the left or right may enlarge or shrink the second graph G2. The operations may also include dragging with a mouse, rotating a mouse wheel, and pressing a predetermined key or button. In addition, as shown in FIG. 10, for example, pinching in or pinching out on the surface of the touch panel laminated on the display 44 with a finger F may reduce or increase the width of the waveform of the second graph G2.

Further, when the waveform of the graph is transformed, the controller 41 transmits a readjustment command signal corresponding to the amount of transformation to the medical image management apparatus 3 via the communication unit 42.

Hence, the readjusted data of the second video I2 is transmitted from the medical image management apparatus 3, and the readjusted second video I2 can be displayed.

In this embodiment, the data of the second video I2 that is the comparative video is adjustable in the medical image management apparatus 3 and the waveform of the second graph G2 corresponding to the second video I2 is therefore transformable. Alternatively, in the case where the medical image management apparatus 3 can adjust the data of the first video I1 or both the data of the first video I1 and the data of the second video I2, the waveform of the first graph G1 may be transformable, or both the waveforms of the first graph G1 and the second graph G2 may be transformable.

In addition, the controller 41 in this embodiment can cause the display 44 to show a graph based on the statistical value generated by the medical image management apparatus 3 as the second graph G2.

To be specific, for example, as shown in FIG. 11, a second graph G2 having the calculated average value as a cycle is displayed on the display 44 or, as shown in FIG. 12, the graph corresponding to the second video I2 having the extracted upper limit signal value is displayed on the display 44 as the second graph G2 and the graph corresponding to the second video I2 having the lower limit signal value is displayed on the display 44 as the third graph G3.

For example, in the medical image management apparatus 3, in the case where the upper limit probability and the lower limit probability are each set to 10%, the first graph G1 falls between the second graph G2 and the third graph G3 with a probability of 80%. In other words, when the first graph G1 falls between the second graph G2 and the third graph G3, the subject has an average motion, and when it extends out from the region sandwiched between the second graph G2 and the third graph G3, there is a possibility that the subject has a motion deviating from the average breathing action that is unlikely to occur stochastically.

Although the specific configuration of the medical image display apparatus 4 according to this embodiment has been described above, at least a part of the aforementioned various functions of the medical image display apparatus 4 may be given to the console 2, the medical image management apparatus 3, and the like.

For example, the medical image display apparatus 4 may have only a browser function, and the medical image management apparatus 3 may have the function of creating a webpage such as the image confirmation screen S.

As described above, in the medical image management system 100 according to this embodiment, the medical image management apparatus 3 constituting the PACS has the function of adjusting the change cycle of the first and second videos I1 and I2, so that the cycles of change of multiple videos obtained by radiographing the cyclic motion of the subject can be adjusted with a simpler configuration.

Further, in a conventional system having the function of adjusting cycles, even if the cycle of the video is adjusted, it cannot be displayed in an effective form (Patent Literature 1 does not concretely mention a difference in the display method and does not clearly show how to compare a diagnostic video to a comparative video).

However, the medical image management system 100 according to this embodiment displays the first and second videos I1 and I2 side by side, or displays the first and second graphs G1 and G2 together with the first and second videos I1 and I2, thereby facilitating comparison between the state of the subject and the same subject in the past or another subject.

Moreover, the medical image management system 100 according to this embodiment can display the first partial video IR1 and the second partial video IR2, thereby enabling more detailed comparison between the state of the subject and the same subject in the past or another subject.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical image management apparatus comprising a first hardware processor that:
acquires a first video obtained by radiographing a cyclic motion of a subject;
acquires a second video obtained by radiographing a cyclic motion of a subject separately from the radiographing of the first video;
extracts a first feature that is a feature of a cyclic change of the acquired first video;
extracts a second feature that is a feature of a cyclic change of the acquired second video;
adjusts at least one of the first video and the second video so that a cycle of change approaches the cycle of change of the other video, based on the extracted first feature and second feature; and
outputs the acquired first video or the adjusted first video, and the acquired second video or the adjusted second video.

2. The medical image management apparatus according to claim 1, wherein
the first video is a main video used for diagnosis,
the second video is a comparative video for comparison with the first video, and
the first hardware processor brings the cycle of the second video close to the cycle of the first video.

3. The medical image management apparatus according to claim 1, wherein the second video is obtained by radiographing the same subject as the subject of the first video in the past, or is obtained by radiographing a subject who is different from the subject of the first video and has a similar physique, a similar age, or the same gender to/as the subject.

4. The medical image management apparatus according to claim 1, wherein the first hardware processor adjusts a part of at least one of the first video and the second video, the part reflecting a change of a second or later period.

5. A medical image management system comprising:
a medical image management apparatus including a first hardware processor that acquires a first video obtained by radiographing a cyclic motion of a subject, acquires a second video obtained by radiographing a cyclic motion of a subject separately from the radiographing of the first video, extracts a first feature that is a feature of a cyclic change of the acquired first video, extracts a second feature that is a feature of a cyclic change of the acquired second video, adjusts at least one of the first video and the second video so that a cycle of change approaches the cycle of change of the other video, based on the extracted first feature and second feature, and outputs the acquired first video or the adjusted first video, and the acquired second video or the adjusted second video; and
a display apparatus including a display and a second hardware processor that causes the display to show the first video acquired by the medical image management apparatus or the first video adjusted by the medical image management apparatus, and the second video acquired by the medical image management apparatus or the second video adjusted by the medical image management apparatus.

6. The medical image management system according to claim 5, wherein the second hardware processor causes the display to show a first graph representing a relationship between time and a signal value in the first video, and causes the display to show a second graph representing a relationship between time and a signal value based on the second video.

7. The medical image management system according to claim 6, wherein
the display apparatus includes an operation unit that can be operated by an operator, and
the second hardware processor
transforms the waveform of at least one of the first graph and the second graph in response to an operation performed on the operation unit, and
readjusts the cycle of change of, of the first video and the second video, the video corresponding to the transformed graph, according to the transformation of at least one of the first graph and the second graph.

8. The medical image management system according to claim 6, further comprising a database that accumulates the second videos, wherein
the first hardware processor generates a statistical value based on the multiple second videos accumulated in the database, and
the second hardware processor causes the display to show a graph based on the statistical value generated by the first hardware processor as the second graph.

9. The medical image management system according to claim 8, wherein
the first hardware processor calculates an average value of each cycle of the multiple second videos based on a second feature extracted from each second video, and
the second hardware processor causes the display to show a second graph having, as a cycle, an average value calculated by the first hardware processor.

10. The medical image management system according to claim 8, wherein
the first hardware processor extracts an upper limit signal value corresponding to a predetermined upper limit reference and a lower limit signal value corresponding to a predetermined lower limit reference in a distribution of predetermined signal values extracted from the multiple second videos, respectively, and
the second hardware processor causes the display to show the second graph corresponding to a second video having the upper limit signal value extracted by the first hardware processor, and causes the display to show a third graph corresponding to a second video having the lower limit signal value.

11. The medical image management system according to claim 5, wherein the second hardware processor extracts a partial region in each frame of the first video as a first region and causes the display to show the partial region as a first partial video, and extracts a partial region corresponding to the first region in each frame of the second video as a second region and causes the display to show the partial region as a second partial video.

12. The medical image management system according to claim 11, wherein
the first video and the second video are radiographic videos of a chest,
the first region is one of a pair of right and left lungs, and
the second region is a lung on a same side as the first region.

13. The medical image management system according to claim 11, wherein
the first video and the second video are radiographic videos of a chest,
the first region is one of a pair of right and left lungs, and
the second region is the lung on an opposite side to the first region.
